(19) United States
(12) Patent Application Publication
(10) Pub. No.: US 2003/0003051 A1
(43) Pub. Date: Jan. 2, 2003

(54) Methods of achieving transplantation tolerance through radioablation of hemolymphopoietic cell populations

(76) Inventors: Luca A. Inverardi, Miami Beach, FL (US); Camillo Ricordi, Miami, FL (US); Giovanni Paganelli, Milano (IT); Aldo N. Serafini, Key Biscayne, FL (US)

Correspondence Address: PILLSBURY WINTHROP, LLP, P.O. BOX 10500, MCLEAN, VA, 22102, US

(21) Appl. No.: 10/166,053

(22) Filed: Jun. 11, 2002

Related U.S. Application Data
(60) Provisional application No. 60/296,723, filed on Jun. 11, 2001.

(51) Int. Cl. A61K 49/00

(52) U.S. Cl. 424/9.1

(57) ABSTRACT

Non-lethal methods of conditioning a recipient prior to bone marrow transplantation to achieve highly enhanced, stable, long-term hematopoietic chimerism in presence of transient immunosuppression are described. In particular, the administration of non-lethal doses of bone-seeking radiopharmaceuticals such as ¹⁵³Samarium Lexidronam, a radioactive compound linked to a tetraphosphonate group, to target bone marrow cells, are disclosed herein.

This application claims the benefit of prior U.S. application Ser. No. 60/296,723, filed Jun. 11, 2001, the entire contents of which are hereby incorporated herein by reference.

### FIELD OF THE INVENTION

The invention relates to the use of radiopharmaceuticals, including but not limited to Samarium, in combination with a variety of conjugates and delivery systems, such as diphosphonates, phosphonates, antibodies, peptides, oligonucleotides or combinations thereof, to target bone marrow cells for therapeutic purposes. These radiopharmaceuticals are particularly useful in inducing chimerism following bone marrow transplantation. The method of the invention has a wide range of application including, but not limited to, conditioning of a recipient prior to hematopoietic reconstitution by bone marrow cell transplantation to treat hematological disorders, hematological malignancies, autoimmune diseases, modulation of the reticulo-endothelial system, infectious diseases and induction of tolerance to solid tissue, cellular, as well as organ grafts.

### BACKGROUND OF THE INVENTION

Transplantation tolerance defined as complete acceptance of a graft by an otherwise fully immunocompetent host without the need for long-term immunosuppression, has been an elusive goal in the field of clinical organ transplantation. Robust tolerance has been achieved in models that made use of bone marrow cell transplantation. Stable multilineage chimerism achieved following bone marrow cell transplantation often has been considered a prerequisite for donor-specific tolerance induction. However, lethal or sub-lethal radiation conditioning strategies commonly used to induce long-term chimerism are often so severely toxic that they preclude the use of these approaches in most clinical conditions other then malignancies or other life-threatening diseases.

Bone marrow transplantation is a commonly utilized procedure for the treatment of hematological disorders including malignancies, and has been recently proposed as a therapeutic option for refractory autoimmune diseases (1, 2, 3, 4, 5, 6, 7). Also, induction of hematopoietic chimerism via bone marrow transplantation results in achievement of donor-specific immunological tolerance allowing successful transplantation of cells, tissues, and solid organs from the bone marrow donors without the need for chronic immunosuppression (8, 9, 10).

Successful induction of transplantation tolerance remains an elusive goal in organ, tissue and cellular transplantation. At present, both chronic and acute graft rejection are alleviated mainly by the use of non-specific immunosuppressive regimens that are often associated with severe complications including development of neoplasms and organ toxicity.

Several models to induce tolerance in animals have been established including achievement of hematopoietic chimerism via bone marrow transplantation. Arguably, tolerance induction using donor bone marrow transplantation resulting in hematopoietic chimerism is the most robust approach to overcoming these problems. This strategy has been shown effective in several animal models where achievement of mixed multilineage chimerism resulted in prolonged survival of donor-derived organs and tissues. However, many tolerance-inducing protocols are based on the use of donor bone marrow infusion following the recipient's treatment with potent cytoreductive (lethal or sub-lethal) conditioning protocols (11 12 13 14), limiting the use of this methodology to the experimental rather then clinical setting.

Many strategies have been used as recipient preconditioning regimens which include the use of lethal and sub-lethal total body irradiation, thymic and /or lymphoid irradiation, as well as the use of cytotoxic drugs, all aiming at the depletion of the recipient hemolymphopoietic cells in order to “make space” for the engraftment of donor-derived elements as well as to induce transient immunosuppression. It has been previously reported that bone marrow has “niches” that support the hematopoietic stem cells via the network of cytokines and growth factors, and that pre-conditioning might create the necessary “space” for the engraftment of donor-derived hematopoietic stem cells (15, 16). In the last few years, the concept of “creating space” by the use of whole body irradiation has been challenged. Rather, single or multiple infusions of large doses of donor bone marrow cells in conjunction with co-stimulatory blockade (anti-CD154, B7, CTLA4-Ig), use of anti-CD4 and anti-CD8 antibodies along with local thymic irradiation have been proposed (17, 18, 19, 20, 21, 22). These approaches, although very promising, still rely on either mega doses of donor-bone marrow cells or some form of external irradiation, methods that would be difficult to implement in the clinical setting.

U.S. Pat. No. 5,273,738 discloses methods utilizing radioactively labeled antibodies in the targeted irradiation of lymphohematopoietic tissue for use in bone marrow rather than particular subsets of cells. This patent does not recognize the importance of chimerism in inducing tolerance.

U.S. Pat. Nos. 5,514,364; 5,635,156; and 5,876,692 describe the use of cell type-specific antibodies directed to antigens localized on subsets of cells in combination with whole body radiation to enhance chimerism and to increase tolerance induction after donor bone marrow transplantation. These patents do not describe the use of non-immunological radioconjugated compounds, such as phosphonate compounds, for the induction of hematopoietic chimerism.

U.S. Pat. No. 5,902,825 (hereinafter the '825 patent) discloses therapeutic compositions containing an active agent complex formed of a non-radioactive metal ion and an organic phosphonic acid ligand, wherein the metal ion may be a Lanthanide. The '825 patent teaches that such compositions may be used in the treatment of bone diseases and in methods of reducing bone pain, but does not address issues related to bone marrow transplantation. In particular, no suggestion is made to therapeutically target bone marrow cells to achieve chimerism via bone marrow transplantation for the induction of tolerance to graft-related antigens.

U.S. Pat. No. 5,697,902 (hereinafter the '902 patent) discloses therapeutic compositions and their methods of use in destroying bone-marrow cells in a patient prior to regrafting with normal bone marrow cells. The disclosed method comprises treating a patient with a cytotoxic amount of an antibody or antibody fragment specific to a marker associated with, or produced by, bone marrow cells and which is conjugated to a cytotoxic agent. According to the '902 patent, suitable antibodies are described as being NP-2, MN3, and other antibodies that react with bone marrow cells, such as progenitor cell types. Radioisotopes preferred for therapeutic use with conjugated antibodies include ¹⁵³Samarium. This patent discloses a protocol for infusion of autologous bone marrow, but does not address the issues concerning successful induction of transplantation tolerance for achieving hematopoietic chimerism via bone marrow transplantation.

U.S. Pat. No. 6,241,961 (hereinafter the '961 patent) discloses therapeutic radioimmunoconjugates for use in human therapy and methods for their production. According to the '961 patent, radioimmunoconjugates may consist of a monoclonal antibody having binding specificity for CD19, CD20, CD22, HLL2, HLA, DR10β, and CD66, conjugated to a radioisotope, and is useful in treating hematopoietic diseases. However, the '961 patent does not suggest the use of non-antibody mediated targeting of bone marrow cells for chimerism induction via bone marrow transplantation for tolerance to alloantigens, autoantigens and xenoantigens.

Therefore, development of suitable protocols that allow the use of low to moderate doses of donor bone marrow inoculum, which do not rely on any form of external irradiation or depletion of the peripheral immune system, is necessary to make the induction of tolerance in bone marrow recipients clinically practical, without invoking harsh preconditioning regimens.

### SUMMARY OF THE INVENTION

The invention focuses on a novel approach of attaining a profound, but transient myelodepression by selectively targeting the recipient bone marrow in order to achieve mixed chimerism. In one embodiment, a series of stable complexes produced as a result of ligating phosphonate derivatives to a number of radioactive compounds have been investigated because of their bone-seeking properties (23). Using this approach, it has become possible to deliver high-energy emitting compounds to a very selective target, in this case, the bone. ¹⁵³Samarium has been found the most promising β- and γ-emitting nucleotide for complexing with phosphonate based on its physical properties. ¹⁵³Samarium (¹⁵³Sm) is a compound with a half-life of 1.9 days. When conjugated to ethylenediaminetetramethylenephosphonate (EDTMP), the radioactive Samarium is characterized by high bone intake and rapid blood clearance (24, 25). Based on these characteristics, the use of ¹⁵³Sm-EDTMP as a palliative treatment of painful bone cancer metastasis has been approved by FDA (26, 27, 28, 29).

Studies performed in both clinical and animal models demonstrated low toxicity and transient myeloablation (23-29). Based on these data, the use of bone-seeking radioactive compounds represents a viable approach to creating the “space” required for the donor hematopoietic stem cells engraftment without the need for external radiation or harsh cytotoxic drugs.

A preferred embodiment of the invention relates to the use of ¹⁵³Sm-diphosphonate conjugates in recipient conditioning in a tolerance-inducing protocol. In particular, ¹⁵³Sm-EDTMP conjugates administered according to the invention induce successful mixed chimerism in recipients as a result of allogeneic bone marrow administration. However, phosphonates, diphosphonates, peptides, and oligonucleotides capable of selectively delivering radioactive Samarium to bone cells are embraced by the inventive method. Such bone specific carriers are known in the art.

Another preferred embodiment is a method of achieving hematopoietic chimerism for induction of immunological tolerance in a recipient of bone marrow transplantation utilizing antibodies that recognize antigens expressed on lymphocytes that participate in cell activation. Methods of inducing mixed chimerism and immunological tolerance according to this embodiment comprise exposing a recipient to a radioimmunoconjugate comprising a radioactive Lanthanide, such as Samarium, conjugated with at least one organic phosphonic acid ligand or a salt thereof. Thereafter, bone marrow cells are transplanted into the recipient via protocols known to those of skill in the art in the presence of at least one antibody raised against an antigen selected from the group consisting of CD4, CD8, CD3, CD5, CD55, CD40, CD40L, B7.1, B7.2, CD28, and LFA-1.

Data generated during the instant studies demonstrates one aspect of the invention, wherein high levels of stable long-term chimerism across a full allogeneic barrier can be achieved by a single administration of a bone seeking radioactive compound, such as ¹⁵³Samarium Lexidronam, prior to the infusion of allogeneic bone marrow cells. For example, allogeneic bone marrow cells may be infused in the presence of a transient T cell co-stimulatory blockade obtained by administration of anti-CD154 monoclonal antibodies (mAb). A large percent of animals tested, followed for up to 31 weeks post bone marrow transplantation, developed donor-specific tolerance, since these animals kept donor-derived skin grafts for more then 150 days.

The data indicate that stable long-term chimerism leading to donor-specific hyporesponsiveness can be achieved without harsh cytotoxic pre-conditioning regimens, and therefore, opens extended possibilities for the use of bone marrow transplantation in a clinical setting. Furthermore, the use of bone-seeking radioactive compounds proven effective in enhancing chimerism levels might prove critical in optimizing strategies to achieve hemopoietic chimerism for the treatment of hematological malignancies and disorders, and autoimmune diseases.

Bone seeking radioactive conjugates according to the invention may be introduced to a human bone marrow recipient in dosages ranging from about 6 mCi/Kg to about 10 mCi/Kg body weight. A single administration of the radioactive complexes should be satisfactory for inducing chimerism following bone marrow transplantation, although multiple dose regimens may be employed, when necessary. Radioactivity will remain in recipient bone, and, therefore, affecting the bone marrow therein, for the life of the isotope. Thus, while radioactive Samarium is preferred, other radioactive isotopes having relatively short, but clinically appropriate, half-lives may also be employed in conjugates according to the invention. Suitable complexes may be prepared in-house according to known protocols optionally utilizing complex forming agents, or may be obtained from commercial sources.

An advantage of the protocols according to the invention over conventional therapies for bone marrow reduction prior to transplantation is the elimination of cumbersome steps required for conjugating radioisotopes to antibodies. Thus, tolerance induction or immunosuppression according to certain preferred embodiments of the invention can be successfully implemented in an efficient manner not previously recognized in the art. In vivo testing of the inventive method using a radioactive conjugate to target bone produced surprising success in inducing myelosuppression in a highly selective manner to achieve chimeris upon bone marrow allotransplantation, as described in the Figures and Example.

### BRIEF DESCRIPTION OF THE DRAWINGS

The invention is further illustrated by the following Figures, wherein:

FIG. 1 graphically depicts the results of treating mice with a single dose, IV, of ¹⁵³Sm-EDTMP, 150 μCi or 500 μCi, prior to administration of 20×10⁶ or 100×10⁶ allogeneic donor bone marrow cells (BMC) as a single intravenous (IV) dose;

FIG. 2 graphically shows that a single administration of BMC resulted in bone marrow engraftment in all recipients analyzed;

FIG. 3 graphically shows the percentage of donor-derived cells in recipients treated with 20×10⁶ BMC, anti-CD154 mAb, and one of 4 conditioning approaches;

FIG. 4 shows the percentage of donor-derived cells in control animals treated with 100×10⁶ BMC and one of the 4 conditioning approaches;

FIG. 5 shows the percentage of donor-derived cells in the control animals treated with 20×10⁶ BMC, and one of the 4 conditioning approaches;

FIG. 6 shows the percent of donor-derived cells in the control animals treated with 20×10⁶ BMC or 100×10⁶ BMC along with anti-CD154 mAb (in the absence of ¹⁵³Sm-EDTMP treatment);

FIG. 7 depicts a two-color flow cytometric analysis of the proportion of donor-derived lymphoid (B cells), NK, and myeloid (granulocytes) lineages in representative mixed chimeras prepared using a non-lethal conditioning regiment of 20×10⁶ BMC, ¹⁵³Sm-EDTMP, and anti-CD154 mAb (upper panels) as well as 20×10⁶ BMC and anti-CD154 mAb (lower panels);

FIG. 8 depicts a two-color flow cytometric analysis of the proportion of donor-derived lymphoid (B cells), NK, and myeloid (granulocytes) lineages in representative mixed chimeras prepared using a non-lethal conditioning regiment of 100×10⁶ BMC, ¹⁵³Sm-EDTMP, and anti-CD154 mAb (upper panels) as well as 100×10⁶ BMC and anti-CD154 mAb (lower panels);

FIG. 9 graphically shows the survival of full thickness tail-derived skin grafts placed on the recipients treated with 20×10⁶ BMC, ¹⁵³Sm-EDTMP, and anti-CD154 mAb, or the indicated control groups; and

FIG. 10 graphically depict the survival of full thickness tail-derived skin grafts placed on the recipients treated with 100×10⁶ BMC, ¹⁵³Sm-EDTMP, and anti-CD154 mAb, or the indicated control groups.

### DETAILED DESCRIPTION OF THE PREFERRED EMBODIMENTS

The invention is further described in the following non-limiting Example.

### EXAMPLE

### Methods

Animals. All animal procedures were performed under the supervision and approval of the University of Miami Institutional Animal Care and Use Committee (IACUC). Mice (7-8 week old Balb/c (H-2^{d}), C57BL/6 (B6; H-2^{b}) and C3H/HeJ (C3H; H-2^{k})) were purchased from Jackson Laboratories (Bar Harbor, Me.). Recipient C57BL/6 mice were used at 9-10 weeks of age. All animals were housed in pathogen-free room in sterile microisolator cages with autoclaved feed and autoclaved acidified water.

Bone marrow transplantation. Balb/c mice, 8-9 weeks old, used as donors, were sacrificed on the day of the transplant. BMC were prepared according to a previously published regimen. Briefly, after removing femura and tibiae, and cleaning them from muscle tissue and cartilage, BMC were flushed with sterile RPMI-1640 (Mediatech, Inc, Herndon, Va.) supplemented with 0.8 mg/ml Gentamycin (Gibco, Gaithersburg, Md.), using 23G needle. BMC were filtered through a sterile nylon mesh and counted. Fully MCH-mismatched C57BL/6 recipients, 9-10 weeks of age, were injected intravenously with either 20×10⁶ or 100×10⁶ unmanipulated BMCs resuspended in 0.5 and 1.0 ml of HBSS (Mediatech) respectively, on either day 7 or 14. Tolerance induction protocol consisted of either 150 or 500 μCi of ¹⁵³Sm-EDTMP (Berlex Laboratories Wayne, N.J.), I.V., on day −7, and 0.5 mg hamster anti-murine CD154 mAb (MR-1), purchased from Taconic (Germantown, N.Y.) administered intraperitoneally (I.P.) on days −1,0,7,14, 21 and 28.

Skin grafting. Full-thickness skin donor (Balb/c) and third party (C3H/HeJ) grafts were transplanted onto the lateral thoracic area of the recipients either the day following BMC-Tx, or 4 weeks following the last administration of MR-1 mAb, using techniques described previously. Briefly, square, full-thickness skin grafts (1 cm²) were prepared from the tail skin of donors. Graft beds were prepared on the right (donor-specific) and left (third party) lateral thoracic wall of recipient mice. Grafts were fixed to the beds with 4 sutures of 5.0 silk at the corners of the graft and covered with a petroleum jelly-coated gauze and a plaster cast. The grafts were first inspected on the eighth-day following grafting, and every third day thereafter. Graft rejection was considered complete when no viable graft tissue was detected by visual inspection. Recipient mice were considered to be tolerant when donor-specific skin grafts survived in perfect condition for <150 days.

Immunohemotyping of chimeras. Engraftment of donor-derived BMCs was ascertained by flow cytometric analysis (FCM) of recipient peripheral blood mononuclear cells (PBMCs), splenocytes, thymocytes and bone marrow cells after staining with FITC-conjugated anti-mouse H-2K^{b} or H-2K^{d} and Cy-Chrome-conjugated CD3 monoclonal antibodies (mAbs) purchased from PharMingen (San Diego, Calif.) at multiple time points during the experiment as well as at sacrifice. Cells were also assessed for non-specific staining using an Ig isotype control (FITC-conjugated mouse IgG₂ₐ and Cy-Chrome-conjugated rat IgG_{2b}), and the percentage of cells stained with this Ab was subtracted from the values obtained from staining with the specific Ab to determine the relative number of positive cells. Reconstitution of various cell lineages was assessed using FITC-conjugated anti-mouse H-2K^{b} or H-2K^{d} and PE-conjugated anti-mouse CD19/CD22 in the B cell, PE-conjugated anti-mouse Ly-6G in the granulocyte, and PE-conjugated anti-mouse Mac-3 in the macrophage compartments. Recipient animals were first tested 1 week after BMC-Tx, every 2 weeks up to 6 weeks, and every 4 weeks thereafter. Purified anti-mouse CD16/CD32 (Fcγ III/II) was used to block non-specific binding to the Fc receptors. FCM analyses were preformed using CellQuest software on a FACScan cytometer purchased from Becton Dickinson & Co. (Mountain View, Calif.).

Analysis of various T cell receptor families. Splenocytes were used to analyze the expression of Vb3⁺, Vb5⁺, Vb11⁺ and Vb14⁺ families in the chimeras at the time of sacrifice. For two-color analysis, cells were blocked with purified anti-mouse CD16/CD32 (Fcγ III/II) (PharMingen), and then incubated with FITC-conjugated H-2K^{d} and PE-conjugated anti-Vb3⁺, Vb5⁺, Vb11⁺ or Vb14⁺ (PharMingen) for 30 minutes on ice. FITC-conjugated mouse IgG2a, PE-conjugated Armenian Hamster IgG, group 2, mouse IgG1, rat IgG2b and rat IgM antibodies (PharMingen) were used as negative controls.

Mixed lymphocyte reaction. Splenocytes depleted of red blood cells were incubated at 37° C. in 5% CO₂ for 3 days in quintuplicate wells containing 2×10⁵ responders with 2×10⁵ stimulators treated with Mytomicin C (Sigma, St. Louis, Mo.) in Iscove's tissue culture media (Gibco, Gaithersburgh, Md.) containing 10% heat-inactivated FCS, 2 mM L-Glutamine (Mediatech), 25 mM HEPES (Mediatech) and 0.05 mM β-mercaptoethanol. Responder cells from chimeric mice and stimulator splenocytes, BMCs and keratinocytes were incubated for 3 days in a 96 round-bottom tissue culture plates, and then pulsed with 1 μCi [³H] thymidine; [³H] thymidine incorporation was assessed after 8 hours. Stimulation indices were calculated by dividing mean counts per minute (c.p.m.) by responses against self.

Staining for the presence of anti-donor antibodies. 1×10⁶ splenocytes, isolated from naïve Balb/c donors were incubated with several different dilutions (1:3; 1:10; 1:30; 1:100) of plasma from the chimeric recipients at 4° C. for 60 minutes. Cells were washed with PBS supplemented with 1% BSA, 0.02% sodium azide, and then incubated with FITC-conjugated goat anti-mouse IgG (H+L) (Jackson ImmunoResearch Laboratories, West Grove, Pa.) and PE-conjugated anti-mouse CD22 for 30 minutes on ice. The cells were then washed with PBS and analyzed on a Becton Dickinson FACScan. Plasma from a naïve C57BL/6 incubated with splenocytes from naïve Balb/c donors was used as a baseline.

### Results

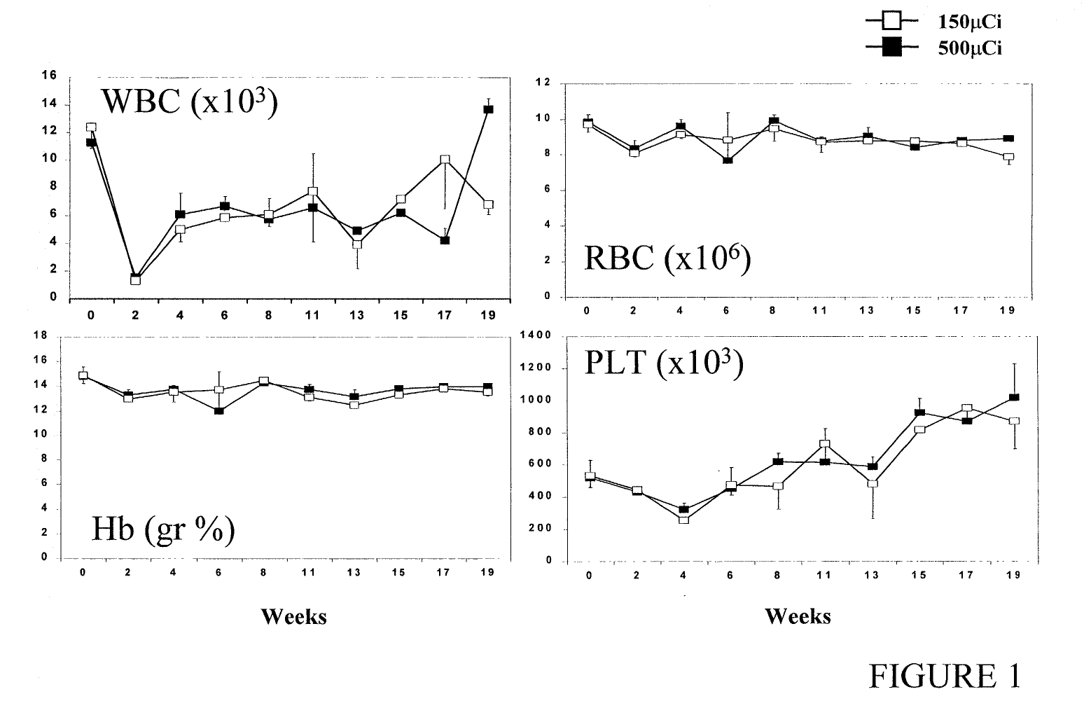

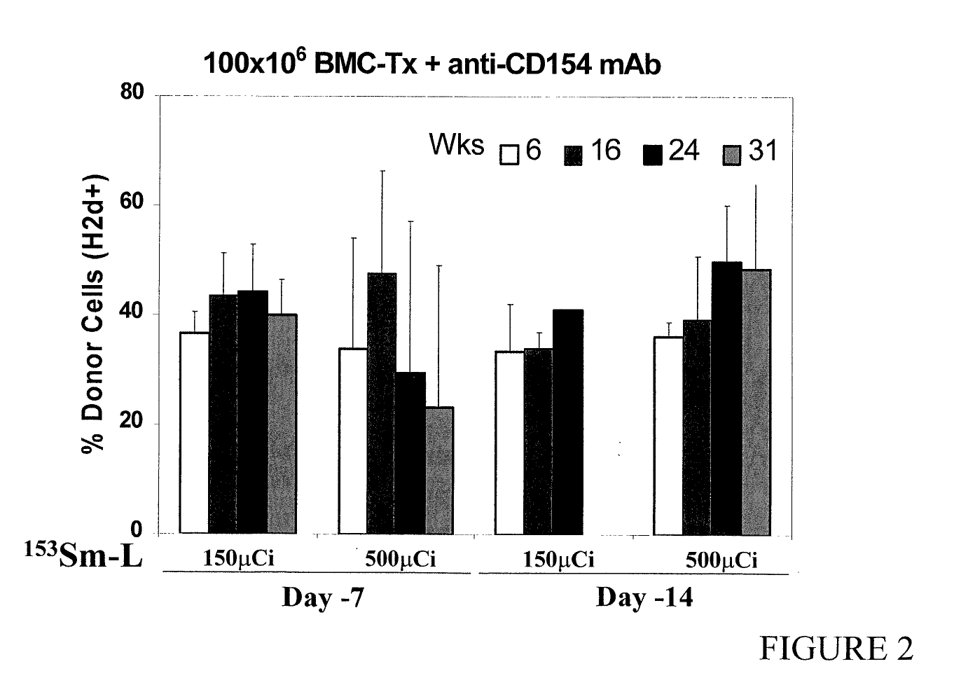

Recipient animals (C57BL/6, H-2^{b}) were treated with a single IV dose of ¹⁵³Sm-EDTMP, 150 μCi or 500 μCi, prior to administration of 20×10⁶ or 100×10⁶ allogenic donor bone marrow cells (BMC) (BALB/c, H-2^{d}), also administered as a single IV dose. BMC transplantation (BMC-Tx) was performed on day 7 or 14 following the administration of ¹⁵³Sm in the presence of transient T lymphocyte co-stimulatory blockade by MR-1 (hamster anti-murine CD154 mAb) on days -1, 0, 7, 14, 21 and 28, 0.5 mg IP. The lower dose of ¹⁵³Sm, 150 μCi, proved to be as effective as the higher dose, 500 μCi. Treatment with ¹⁵³Sm-EDTMP resulted in transient myelodepression that occurred one week post administration of the compound and was spontaneously resolved by 4-6 weeks post-administration, as shown in FIG. 1. Both the 150 μCi and 500 μCi doses of ¹⁵³Sm-EDTMP have similar effect on hemolymphopoietic elements. Although there is a marked myelodepression, as assessed by a decreased white blood cell counts (WBC), administration of ¹⁵³Sm-EDTMP does not have significant effect on red blood cell (RBC), hemoglobin (Hb), and Platelet (PLT) counts. Similar data were obtained in animals treated with ¹⁵³Sm-EDTMP and not transplanted with allogeneic BMC (not shown). Thus, ¹⁵³Sm-EDMP leads to a transient myelodepression of the WBC compartment, which is spontaneously reversible either in the presence or absence of an allogeneic BMC-Tx. No dramatic alterations of RBC, PLT or Hb counts were evident. Single administration of BMC resulted in BM engraftment in all recipient animals analyzed. FIG. 2 shows percentages of donor-derived cells in the recipients treated with 100×10⁶ BMC, anti-CD154 mAb, and one of 4 conditioning approaches—¹⁵³Sm-EDTMP 150 μCi, followed by administration of BMC on day 7; ¹⁵³Sm-EDTMP 500 μCi, followed by administration of BMC on day 7, ¹⁵³Sm-EDTMP 150 μCi, followed by administration of BMC on day 14; and ¹⁵³Sm-EDTMP 500 μCi, followed by administration of BMC on day 14. Typing of PBL obtained from the recipient animals starting at 2 weeks following the reconstitution with donor-derived BM allogeneic cells, every two weeks up to 6 weeks post-reconstitution, and every 4 weeks afterwards was performed using anti Class I H-2^{b}-FITC and H-2^{d}-FITC. Analysis was performed on the lymphoid gate, and the values were normalized to 100%. CD3+T lymphocytes of donor origin were also present, suggesting mixed chimerism of the lymphoid lineage as well.

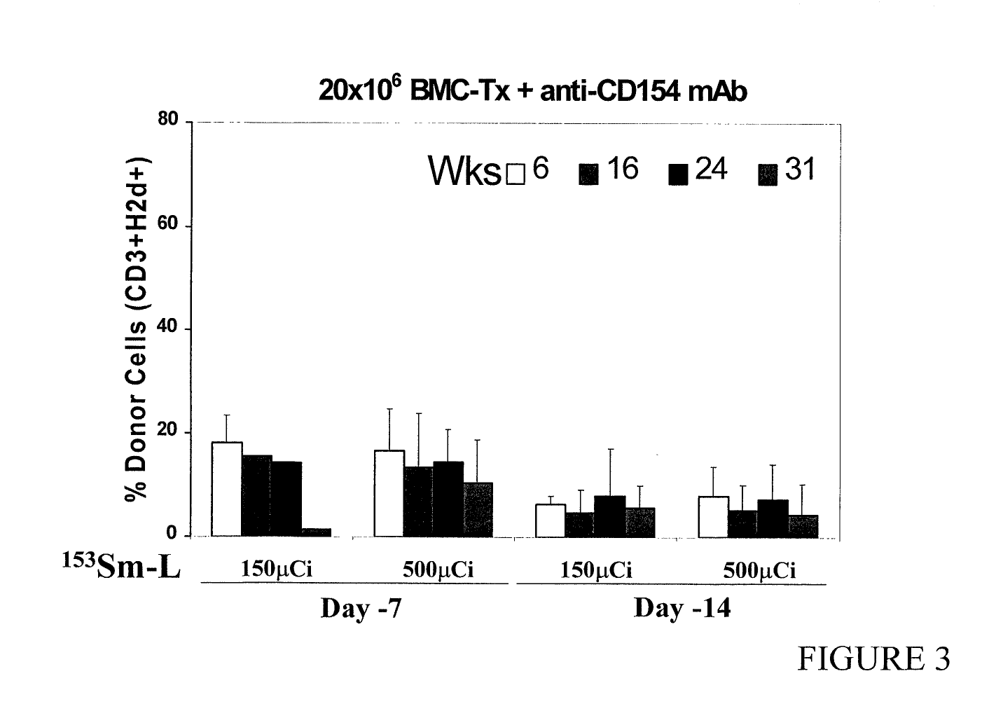

In FIG. 3 is shown the percentage of donor-derived cells in the recipients treated with 20×10⁶ BMC, anti-CD154 mAb, and one of the 4 conditioning approaches: ¹⁵³Sm-EDTMP 150 μCi, followed by administration of BMC on day 7; ¹⁵³Sm-EDTMP 500 μCi, followed by administration of BMC on day 7; ¹⁵³Sm-EDTMP 150 μCi, followed by administration of BMC on day 14; and ¹⁵³Sm-EDTMP 500 μCi, followed by administration of BMC on day 14. Typing of PBL obtained from the recipient animals starting at 2 weeks following the reconstitution with donor-derived BM allogeneic cells, every two weeks up to 6 weeks post-reconstitution, and every 4 weeks afterwards was performed using anti Class I H-2^{b}-FITC and H-2^{d}-FITC. Analysis was performed on the lymphoid gate, and the values were normalized to 100%. CD3+T lymphocytes of donor origin were also present, suggesting mixed chimerism of the lymphoid lineage as well.

Therefore, administration of ¹⁵³Sm-EDMP in the presence of costimulatory blockade leads to long-lasting hematopoietic chimerism in the recipients of allogeneic BMC. The dose of ¹⁵³Sm-EDMP (150 μCi vs. 500 μCi) and the timing of BMC-Tx relative to ¹⁵³Sm-EDMP administration do not grossly influence the results. BMC dose, on the other hand, directly correlates with the levels of chimerism achieved.

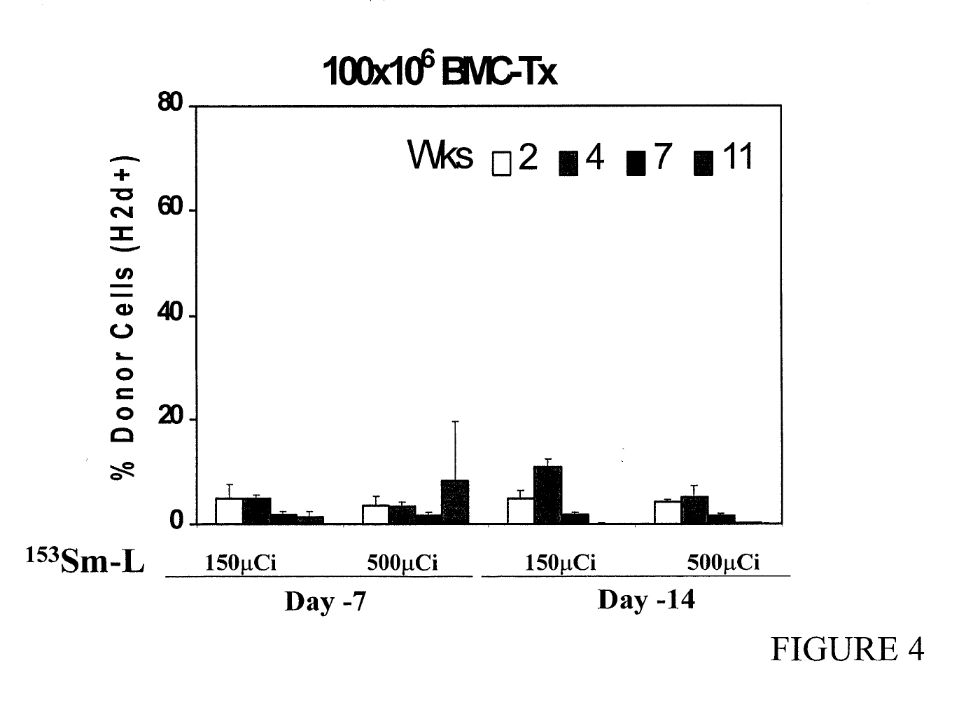

As shown in FIG. 4, the percentage of donor-derived cells in the control animals treated with 100×10⁶ BMC and one of the 4 conditioning approaches was assessed. The conditioning regimens were ¹⁵³Sm-EDTMP 150 μCi, followed by administration of BMC on day 7; ¹⁵³Sm-EDTMP 500 μCi, followed by administration of BMC on day 7; ¹⁵³Sm-EDTMP 150 μCi, followed by administration of BMC on day 14; and ¹⁵³Sm-EDTMP 500 μCi, followed by administration of BMC on day 14. This fourth regimen differs from the previous, since no anti-CD154 mAb to induce costimulatory blockade was used. Typing of PBL obtained from the recipient animals starting at 2 weeks following the reconstitution with donor-derived BMC allogeneic cells, every two weeks up to 6 weeks post-reconstitution, and every 4 weeks afterwards was performed using anti Class I H-2^{b}-FITC and H-2^{d}-FITC. Analysis was performed on the lymphoid gate, and the values were normalized to 100%.

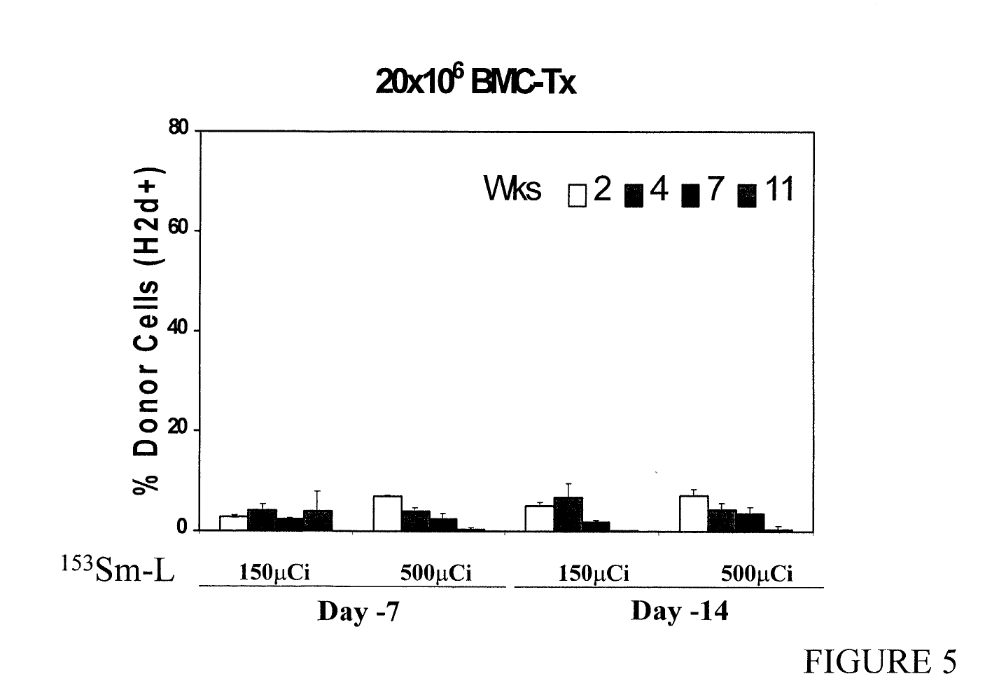

FIG. 5 shows the percent of donor-derived cells in the control animals treated with 20×10⁶ BMC, and one of the 4 conditioning approaches: ¹⁵³Sm-EDTMP 150 μCi, followed by administration of BMC on day 7; ¹⁵³Sm-EDTMP 500 μCi, followed by administration of BMC on day 7; ¹⁵³Sm-EDTMP 150 μCi, followed by administration of BMC on day 14; and ¹⁵³Sm-EDTMP 500 μCi, followed by administration of BMC on day 14 (this regimen differs from the previous, since no anti-CD154 mAb to induce costimulatory blockade was used). Typing of PBL obtained from the recipient animals starting at 2 weeks following the reconstitution with donor-derived BMC allogeneic cells, every two weeks up to 6 weeks post-reconstitution, and every 4 weeks following that was performed using anti Class I H-2^{b}-FITC and H-2^{d}-FITC. Analysis was performed on the lymphoid gate, and the values were normalized to 100%.

Thus, the data from FIGS. 4-5 show that in the absence of co-stimulatory blockade, ¹⁵³Sm-EDMP administration followed by BMC-Tx only leads to transient chimerism, regardless of the dose of BMC (20×10⁶ or 100×10⁶).

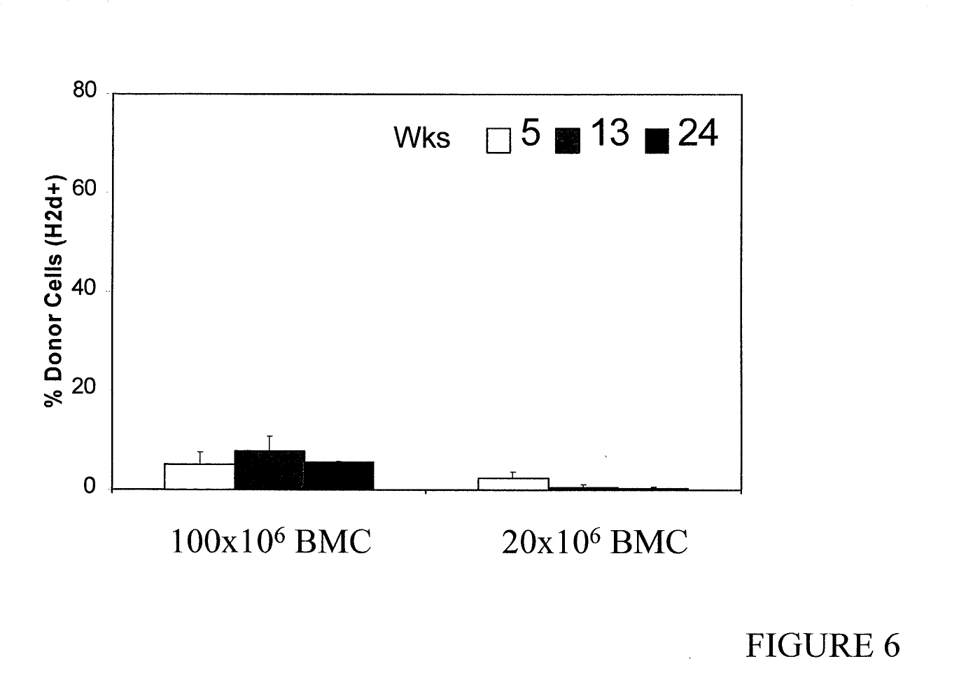

The percentage of donor-derived cells in the control animals treated with 20×10⁶ BMC or 100×10⁶ BMC along with anti-CD154 mAb (in the absence of ¹⁵³Sm-EDTMP treatment) is shown in FIG. 6. Typing of PBL obtained from the recipient animals starting at 2 weeks following the reconstitution with donor-derived BMC allogeneic cells, every two weeks up to 6 weeks post-reconstitution, and every 4 weeks following that was performed using anti Class I H-2^{b}-FITC and H-2^{d}-FITC. Analysis was performed on the lymphoid gate, and the values to were normalized to 100%. The results indicate that treatment with BMC-Tx and co-stimulatory blockade without administration of ¹⁵³Sm-EDMP, leads to transient chimerism when a low dose (20×10⁶) BMC is administered and to low level, stable chimerism when 100×10⁶ BMC are administered.

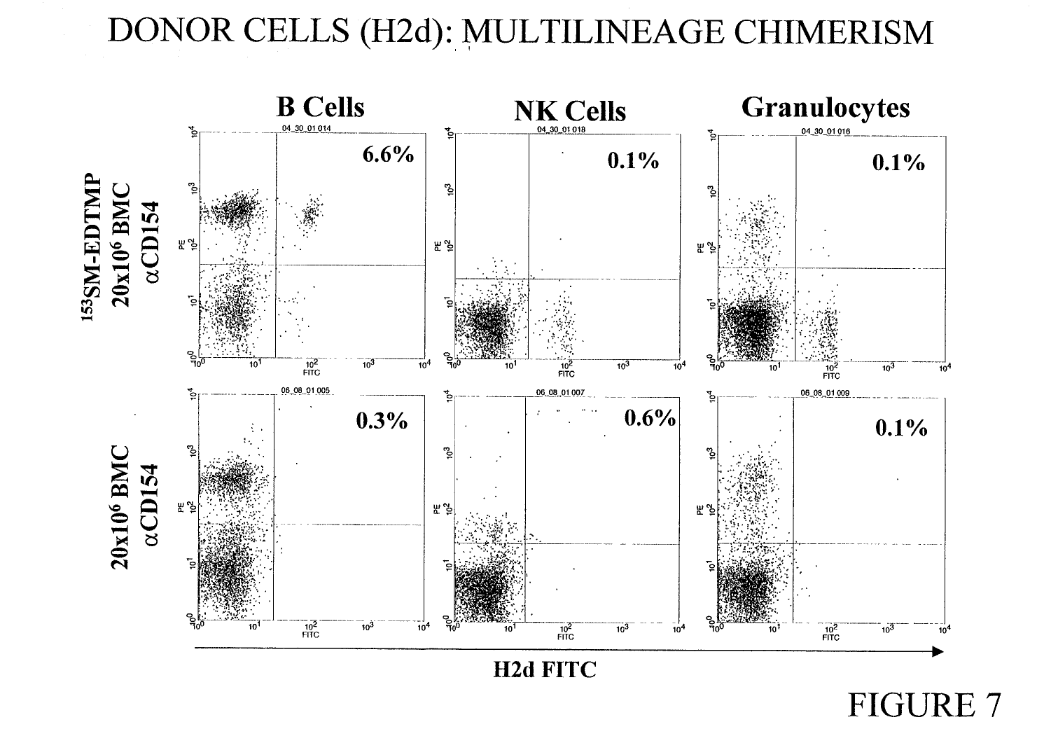

FIG. 7 shows a two-color flow cytometric analysis of the proportion of donor-derived lymphoid (B cells), NK, and myeloid (granulocytes) lineages in representative mixed chimeras prepared using a non-lethal conditioning regiment of 20×10⁶ BMC, ¹⁵³Sm-EDTMP, and anti-CD154 mAb (upper panels) as well as 20×10⁶ BMC and anti-CD154 mAb (lower panels). Analysis was performed using Class I H-2^{d}-FITC and either CD22 (B cells), NK, or GRAN1 (granulocytes), all PE. Analysis was performed on the lymphoid gate, and the values were normalized to 100%.

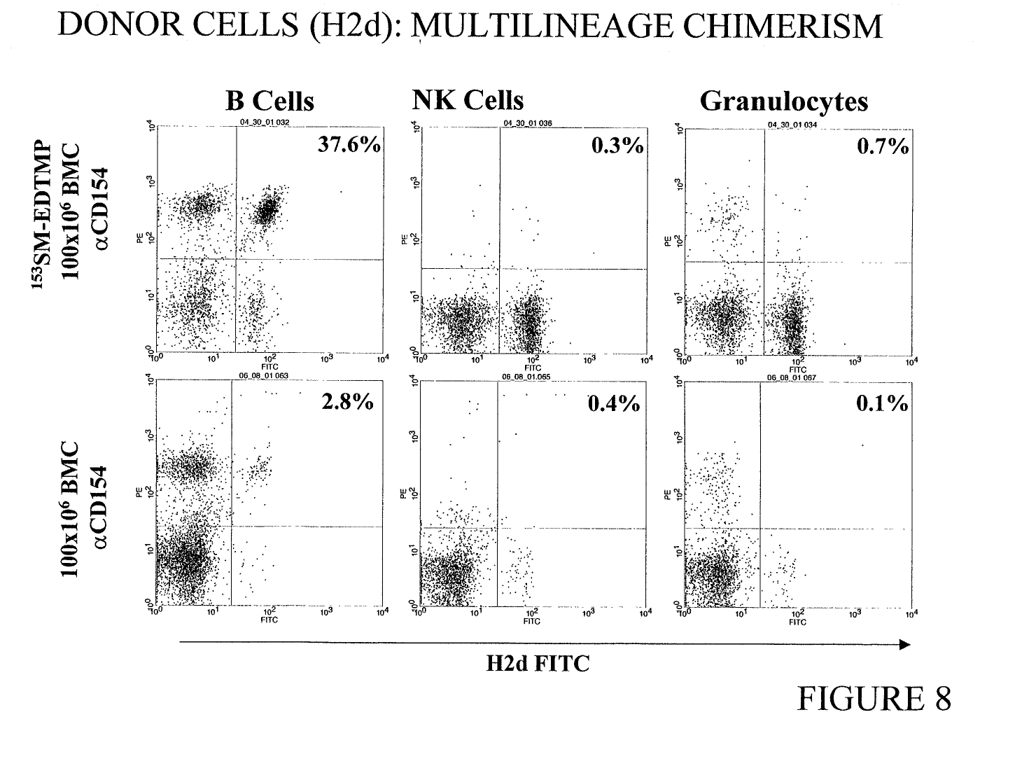

In FIG. 8 is shown a two-color flow cytometric analysis of the proportion of donor-derived lymphoid (B cells), NK, and myeloid (granulocytes) lineages in representative mixed chimeras prepared using a non-lethal conditioning regiment of 100×10⁶ BMC, ¹⁵³Sm-EDTMP, and anti-CD154 mAb (upper panels) as well as 100×10⁶ BMC and anti-CD154 mAb (lower panels). Analysis was preformed using Class I H-2^{d}-FITC and either CD22 (B cells), NK, or GRAN1 (granulocytes), all PE. Analysis was performed on the lymphoid gate, and the values were normalized to 100%.

As is evident from the data presented in FIGS. 7 and 8, long-term, stable multilineage chimerism is achieved in the group treated with a combination of BMC-Tx, ¹⁵³Sm-EDMP, and anti-CD154 mAb.

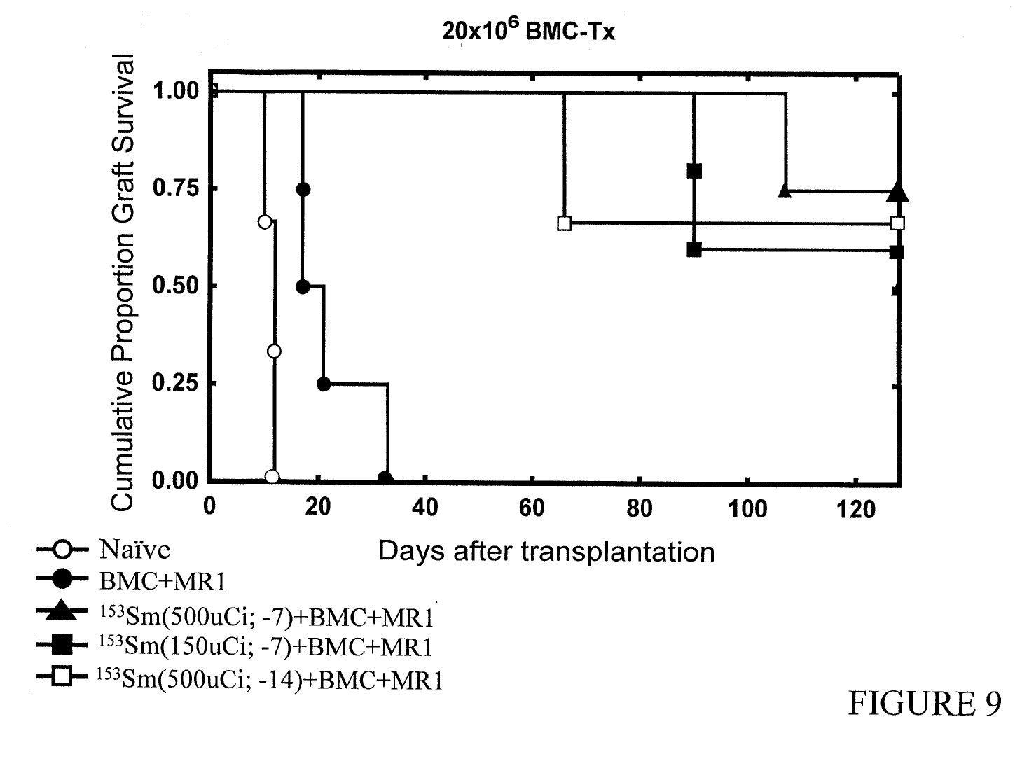

The survival of full thickness tail-derived skin grafts placed on the recipients treated with 20×10⁶ BMC, ¹⁵³Sm-EDTMP, and anti-CD154 mAb, or indicated control groups is shown in FIG. 9. Grafts were prepared 30 days following the last administration of anti-CD154 mAb in the treated animals. Two different donor strain combinations, BALB/c (H-2^{d}) and C3H/J (H-2^{k}) were used. Each recipient received skin grafts from both strains: donor-type, BALB/c (H-2^{d}), as well as third-party, C3H/J (H-2^{k}). Third party grafts were rejected within the same time frame as were donor-specific grafts placed on naive recipients. Grafts were followed for a minimum of 128 days and were considered rejected when viable tissue was no longer detected at the transplant site. Therefore, tolerance to donor-specific skin grafts is obtained when animals receive a low dose of BMC (20×10⁶), only if ¹⁵³Sm-EDMP is part of the treatment, while co-stimulation alone (along with BMC) is not sufficient to achieve the same result.

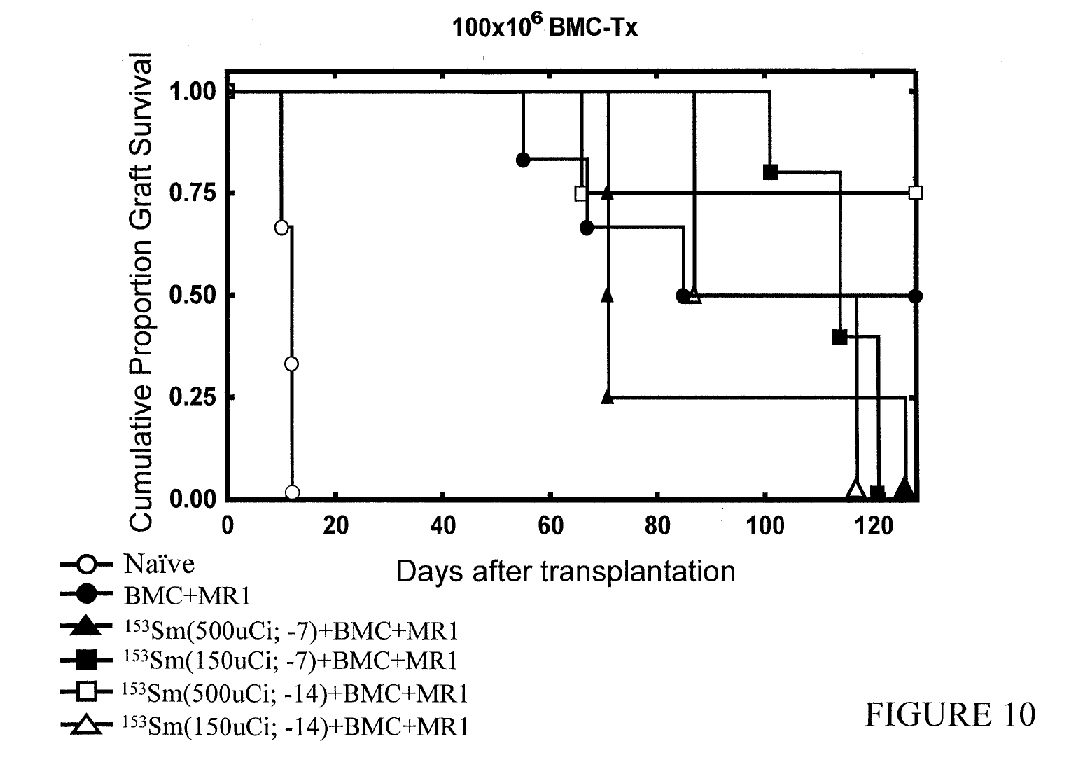

The survival of full thickness tail-derived skin grafts placed on the recipients treated with 100×10⁶ BMC, ¹⁵³Sm-EDTMP, and anti-CD154 mAb, or indicated control groups is depicted graphically in FIG. 10. Grafts were prepared 30 days following the last administration of anti-CD154 mAb in the treated animals. Two different donor strain combinations, BALB/c (H-2^{d}) and C3H/J (H-2^{k}) were used. Each recipient received skin grafts from both strains: donor-type, BALB/c (H-2^{d}), as well as third-party, C3H/J (H-2^{k}). Third party grafts were rejected within the same time frame as were donor-specific grafts placed on naïve recipients. Grafts were followed for a minimum of 128 days and were considered rejected when viable tissue was no longer detected at the transplant site. Thus, when a high dose of BMC is given (100×10⁶), the enhancing effect of ¹⁵³Sm-EDMP administration is still visible on chimerism levels, that are reproducibly higher, but lost on graft survival since co-stimulatory blockade only (+BMC-Tx) appears similarly efficacious.

Radionuclide complexes between lanthanides and bone specific carriers may be formulated into any pharmaceutically acceptable dosage form, including liquids, emulsions, suspensions and the like. Liquid solutions for injection are particularly preferred. Pharmaceutical compositions of the complexes for use according to the invention may also contain suitable diluents, excipients, buffers, stabilizers and carriers. Sterile water or sterile isotonic saline solutions are particularly preferred.

While the invention has been illustrated via the preferred embodiments described above, it will be understood that the invention may be practiced employing various modifications evident to those skilled in the art without departing from the spirit and scope of the invention as generally described herein, and as further set forth by the appended claims.

### REFERENCES

1. Saba N, Flaig T. Bone marrow transplantation for nonmalignant diseases. J Hematother Stem Cell Res. 2002 (2):377-87.

2. Furst D E. Stem cell transplantation for autoimmune disease: progress and problems. Curr Opin Rheumatol. 2002;14(3):220-4.

3. Oyama Y, Papadopoulos E B, Miranda M, Traynor A E, Burt R K. Allogeneic stem cell transplantation for Evans syndrome. Bone Marrow Transplant. 2001;28(9):903-5.

4. Pratt G, Kinsey S E.Remission of severe, intractable autoimmune haemolytic anaemia following matched unrelated donor transplantation.Bone Marrow Transplant. 2001;28(8):791-3.

5. Berdeja J G, Flinn I W. New approaches to blood and marrow transplantation for patients with low-grade lymphomas. Curr Opin Oncol. 2001;13(5):335-41.

6. Chilton P M, Huang Y, Ildstad S T. Bone marrow cell graft engineering: from bench to bedside. Leuk Lymphoma. 2001;41(1-2):19-34.

7. Burt R K, Slavin S, Burns W H, Marmont A M. Induction of tolerance in autoimmune diseases by hematopoietic stem cell transplantation: getting closer to a cure? Blood. 2002;99(3):768-84.

8. Inverardi L, Ricordi C. Tolerance and pancreatic islet transplantation. Philos Trans R Soc Lond B Biol Sci. 2001;356(1409):759-65

9. Waldmann H. Therapeutic approaches for transplantation. Curr Opin Immunol. 2001;13(5):606-10.

10. Sykes M, Sachs D H. Mixed chimerism. Philos Trans R Soc Lond B Biol Sci. 2001;356(1409):707-26.

11. Mayumi H, Good R A. Induction of tolerance across major barriers using a two-step method with genetic analysis of tolerance induction.Immunobiology. 1989;179(1):86-108.

12. Ildstad S T, Sachs D H. Reconstitution with syngeneic plus allogeneic or xenogeneic bone marrow leads to specific acceptance of allografts or xenografts. Nature. 1984;307(5947):168-70.

13. Sharabi Y, Sachs D H. Mixed chimerism and permanent specific transplantation tolerance induced by a nonlethal preparative regimen. J Exp Med. 1989;169(2):493-502.

14. Colson Y L, Li H, Boggs S S, Patrene K D, Johnson P C, Ildstad S T. Durable mixed allogeneic chimerism and tolerance by a nonlethal radiation-based cytoreductive approach. J Immunol. 1996;157(7):2820-9.

15. Stewart F M, Crittenden R B, Lowry P A, Pearson-White S, Quesenberry P J.Long-term engraftment of normal and post-5-fluorouracil murine marrow into normal nonmyeloablated mice.Blood. 1993;81(10):2566-71.

16. Rao S S, Peters S O, Crittenden R B, Stewart F M, Ramshaw H S, Quesenberry P J.Stem cell transplantation in the normal nonmyeloablated host: relationship between cell dose, schedule, and engraftment.Exp Hematol. 1997;25(2):114-21.

17. Durham M M, Bingaman A W, Adams A B, Ha J, Waitze S Y, Pearson T C, Larsen C P. Cutting edge: administration of anti-CD40 ligand and donor bone marrow leads to hemopoietic chimerism and donor-specific tolerance without cytoreductive conditioning. J Immunol. 2000;165(1):1-4.

18. Pearson T C, Alexander D Z, Hendrix R, Elwood E T, Linsley P S, Winn K J, Larsen C P. CTLA4-Ig plus bone marrow induces long-term allograft survival and donor specific unresponsiveness in the murine model. Evidence for hematopoietic chimerism. Transplantation. 1996;61(7): 997-1004.

19. Seung E, Iwakoshi N, Woda B A, Markees T G, Mordes J P, Rossini A A, Greiner D L. Allogeneic hematopoietic chimerism in mice treated with sublethal myeloablation and anti-CD154 antibody: absence of graft-versus-host disease, induction of skin allograft tolerance, and prevention of recurrent autoimmunity in islet-allografted NOD/Lt mice. Blood. 2000;95(6):2175-82.

20. Wekerle T, Kurtz J, Ito H, Ronquillo J V, Dong V, Zhao G, Shaffer J, Sayegh M H, Sykes M. Allogeneic bone marrow transplantation with co-stimulatory blockade induces macrochimerism and tolerance without cytoreductive host treatment. Nat Med. 2000;6(4):464-9.

21. Wekerle T, Sayegh M H, Ito H, Hill J, Chandraker A, Pearson D A, Swenson K G, Zhao G, Sykes M. Anti-CD154 or CTLA4Ig obviates the need for thymic irradiation in a non-myeloablative conditioning regimen for the induction of mixed hematopoietic chimerism and tolerance. Transplantation. 1999;68(9):1348-55.

22. Sharabi Y, Abraham V S, Sykes M, Sachs D H. Mixed allogeneic chimeras prepared by a non-myeloablative regimen: requirement for chimerism to maintain tolerance. Bone Marrow Transplant. 1992;9(3):191-7.

23. Goeckeler W F, Edwards B, Volkert W A, Holmes R A, Simon J, Wilson D. Skeletal localization of samarium-153 chelates: potential therapeutic bone agents. J Nucl Med. 1987;28(4):495-504.

24. Goeckeler W F, Edwards B, Volkert W A, Holmes R A, Simon J, Wilson D. Skeletal localization of samarium-153 chelates: potential therapeutic bone agents. J Nucl Med. 1987;28(4):495-504.

25. Turner J H, Claringbold P G, Berger J D, Martindale A A, Glancy J R. ¹⁵³Sm-EDTMP and melphalan chemoradiotherapy regimen for bone marrow ablation prior to marrow transplantation: an experimental model in the rat. Nucl Med Commun. 1992;13(5):321-9.

26. Serafini A N. Systemic metabolic radiotherapy with samarium-153 EDTMP for the treatment of painful bone metastasis. Q J Nucl Med. 2001;45(1):91-9.

27. Serafini A N, Houston S J, Resche I, Quick D P, Grund F M, Ell P J, Bertrand A, Ahmann F R, Orihuela E, Reid R H, Lerski R A, Collier B D, McKillop J H, Purnell G L, Pecking A P, Thomas F D, Harrison K A. Palliation of pain associated with metastatic bone cancer using samarium-153 lexidronam: a double-blind placebo-controlled clinical trial. J Clin Oncol. 1998;16(4):1574-81.

28. Farhanghi M, Holmes R A, Volkert W A, Logan K W, Singh A. Samarium-153-EDTMP: pharmacokinetic, toxicity and pain response using an escalating dose schedule in treatment of metastatic bone cancer. J Nucl Med. 1992;33(8):1451-8.

28. Collins C, Eary J F, Donaldson G, Vernon C, Bush N E, Petersdorf S, Livingston R B, Gordon E E, Chapman C R, Appelbaum F R. Samarium-153-EDTMP in bone metastases of hormone refractory prostate carcinoma: a phase I/II trial. J Nucl Med. 1993 Nov;34(11):1839-44.

What is claimed is:

1. A method of achieving hematopoietic chimerism for induction of immunological tolerance in a recipient of bone marrow transplantation comprising:
exposing a recipient to a radioimmunoconjugate comprising a radioactive Samarium compound conjugated with at least one member of the group consisting of diphosphonates, phosphonates, peptides and oligonucleotides; and
transplanting bone marrow cells into the recipient.

2. The method according to claim 1, wherein the immunological tolerance comprises tolerance to at least one member of the group consisting of alloantigens, autoantigens and xenoantigens.

3. The method according to claim 1, wherein the radioimmunoconjugate is administered in a single dosage ranging between about 6 mCi/Kg to about 10 mCi/kg body weight.

4. The method according to claim 3, wherein the radioimmunoconjugate is administered intravenously.

5. The method according to claim 1, wherein the radioactive Samarium compound is conjugated to ethylenediaminetetramethylenephosphonate.

6. The method according to claim 5, wherein the radioactive Samarium compound is ¹⁵³Samarium Lexidronam.

7. The method according to claim 1 further comprising transplanting bone marrow cells into the recipient in the presence of at least one antibody that recognizes antigens expressed on lymphocytes that participate in cell activation.

8. The method according to claim 7, wherein the at least one antibody recognizes an antigen selected from the group consisting of CD4, CD8, CD3, CD5, CD55, CD40, CD40L, B7.1, B7.2, CD28, and LFA-1.
